(19) United States
(12) Patent Application Publication
(10) Pub. No.: US 2022/0225445 A1
(43) Pub. Date: Jul. 14, 2022

(54) REMOTE MONITORING OF A SORBENT RECHARGER

(71) Applicant: Medtronic, Inc., Minneapolis, MN (US)

(72) Inventor: Sharath Nagendra, Bangalore (IN)

(73) Assignee: Medtronic, Inc, Minneapolis, MN (US)

(21) Appl. No.: 17/148,900

(22) Filed: Jan. 14, 2021

(51) Int. Cl. H04W 76/14; H04W 4/80; H04W 12/06; A61M 1/16

(52) U.S. Cl. CPC H04W 76/14; A61M 1/1601; H04W 12/06; H04W 4/80

(57) ABSTRACT

The disclosure relates to systems and methods for remotely controlling one or more sorbent rechargers. The systems and methods can include at least one remote device in communication with at least one sorbent recharger. The remote device can control at least one function necessary for the sorbent rechargers to recharge sorbent materials in reusable sorbent modules.

### FIELD

The disclosure relates to systems and methods for remotely controlling one or more sorbent rechargers. The systems and methods can include at least one remote device in communication with at least one sorbent recharger. The remote device can control at least one function necessary for the sorbent rechargers to recharge sorbent materials in reusable sorbent modules.

### BACKGROUND

Sorbent materials are used in sorbent-based hemodialysis to remove solutes from used dialysate, allowing the dialysate to be reused. In order to reuse the sorbent materials, the sorbent materials must be recharged after every use. To recharge the sorbent materials, a sorbent recharger can perform a process by which chemicals are pumped through a sorbent material within a reusable sorbent module. To control or provide instructions to the sorbent recharger, to monitor a recharging process, or to recover any errors or alerts on the sorbent recharger, a user must provide information or data to the sorbent recharger.

Each time a user provides information or data to a sorbent recharger, the user must come near to and touch the sorbent recharger. In a clinical setting, direct contact between a clinician and a sorbent recharger can increase the likelihood of contamination between multiple patients. Further, requiring direct contact between a user and the sorbent recharger can increase the time necessary for recharging sorbent materials and requires the user to be physically present during the entire process.

Hence, there is a need for systems and methods that allow a user or clinician to remotely operate one or more sorbent rechargers, minimizing direct contact between the users and the sorbent rechargers. The need extends to systems and methods for setting recharger configuration parameters, monitoring the processes, and taking actions in response to alerts or errors.

### SUMMARY OF THE INVENTION

The problem to be solved by the present invention is controlling one or more sorbent rechargers and the process of recharging by the sorbent rechargers while minimizing contact between users and the sorbent rechargers and decreases the time spent performing functions necessary for recharging sorbent materials. The solution is to include one or more remote devices that can establish communication with one or more sorbent rechargers and control at least one function necessary for recharging the sorbent materials.

The first aspect of the invention relates to a system. In any embodiment, the system can include at least one sorbent recharger; and at least one remote device in communication with the at least one sorbent recharger; the at least one remote device programmed to control at least one function necessary for the sorbent recharger to recharge a sorbent material in a reusable sorbent module.

In any embodiment, the at least one remote device can be a wearable device.

In any embodiment, the system can include at least a second sorbent recharger in communication with the at least one remote device; the at least one remote device programmed to control at least one function necessary for the second sorbent recharger to recharge a sorbent material in a second reusable sorbent module.

In any embodiment, the system can include at least a second remote device in communication with the at least one sorbent recharger; the second remote device programmed to control at least one function necessary for the at least one sorbent recharger to recharge the sorbent material.

In any embodiment, the system can include at least a second remote device and a second sorbent recharger; the second the second remote device programmed to control at least one function necessary for the second sorbent recharger to recharge a sorbent material in a second reusable sorbent module.

In any embodiment, each remote device can be in communication with each sorbent recharger; wherein each remote device can be programmed to control at least one function necessary for each sorbent recharge to recharge the sorbent material in each reusable sorbent module.

The features disclosed as being part of the first aspect of the invention can be in the first aspect of the invention, either alone or in combination, or follow any arrangement or permutation of any one or more of the described elements. Similarly, any features disclosed as being part of the first aspect of the invention can be in a second or a third aspect of the invention described below, either alone or in combination, or follow any arrangement or permutation of any one or more of the described elements.

The second aspect of the invention relates to a method performed by a remote device. In any embodiment, the method can include the steps of: establishing communication between the remote device and at least one sorbent recharger; and controlling at least one function necessary for the at least one sorbent recharger to recharge a sorbent material in a reusable sorbent module.

In any embodiment, the method can include the step of authenticating the sorbent recharger and transmitting remote device authentication information to the recharger.

In any embodiment, the method can include the step of displaying data from the sorbent recharger on a display.

In any embodiment, the data received from the sorbent recharger can include one or more of: a recharge status, an alert, a request for action, and/or recharger configuration information.

In any embodiment, the method can include the step of providing an audio and/or a haptic signal based on data received from the sorbent recharger.

In any embodiment, the method can include the step of receiving data from a user and transmitting the data to the sorbent recharger.

In any embodiment, the data from the user can include at least one of: an action to recover an error on the sorbent recharger, an action to recover an alert from the sorbent recharger, an action to initiate a function in recharging the sorbent material, an action to stop recharging the sorbent material, an action to power off the sorbent recharger, an action to initiate one or more diagnostic tests, and/or an action to set or change one or more configuration parameters for the recharger.

In any embodiment, the method can include the steps of establishing communication between the remote device and at least a second sorbent recharger; and controlling at least one function necessary for the second sorbent recharger to recharge a sorbent material in a reusable sorbent module.

The features disclosed as being part of the second aspect of the invention can be in the second aspect of the invention, either alone or in combination, or follow any arrangement or permutation of any one or more of the described elements. Similarly, any features disclosed as being part of the second aspect of the invention can be in the first aspect of the invention or a third aspect of the invention, either alone or in combination, or follow any arrangement or permutation of any one or more of the described elements.

The third aspect of the invention relates to a remote device. In any embodiment the remote device can include a processor programmed to: establish wireless communication with at least a first sorbent recharger; and to control at least one function necessary for the sorbent recharger to recharge a sorbent material in a reusable sorbent module.

In any embodiment, the remote device can be a wearable device.

In any embodiment, the remote device can be programmed to communicate with the sorbent recharger via Bluetooth, Wi-Fi, and/or Wi-Fi direct.

In any embodiment, the remote device can include a display, the remote device programmed to display data received from the sorbent recharger on the display.

In any embodiment, the remote device can be programmed to receive one or more configuration parameters from the sorbent recharger and to display the one or more configuration parameters on the display.

In any embodiment, the data received from the sorbent recharger can include recharge status, an alert, and/or a request for action.

In any embodiment, the remote device can include an input, the remote device programmed to transmit data from the input to the sorbent recharger.

In any embodiment, the data from the input can include at least one of: an action to recover an error on the sorbent recharger, an action to recover an alert from the sorbent recharger, an action to initiate a function in recharging the sorbent material, an action to stop recharging the sorbent material, an action to power off the sorbent recharger, an action to initiate one or more diagnostic tests, and/or an action to set or change one or more configuration parameters for the sorbent recharger.

In any embodiment, the remote device can be programmed to establish wireless communication and control at least one function of at least a second sorbent recharger.

In any embodiment, the remote device can be programmed to receive sorbent recharger authentication information from the sorbent recharger and to transmit remote device authentication information to the recharger.

In any embodiment, the remote device can be programmed to provide an audio and/or a haptic signal based on data received from the sorbent recharger.

In any embodiment, the data received from the recharger can include one or more alerts.

The features disclosed as being part of the third aspect of the invention can be in the third aspect of the invention, either alone or in combination, or follow any arrangement or permutation of any one or more of the described elements. Similarly, any features disclosed as being part of the third aspect of the invention can be in the first or second aspect of the invention, either alone or in combination, or follow any arrangement or permutation of any one or more of the described elements.

### BRIEF DESCRIPTION OF THE DRAWINGS

FIG. 1 is diagram showing a remote device for controlling a sorbent recharger.

FIG. 2 shows use of a single remote device to control a single sorbent recharger by a user.

FIG. 3 shows use of a single remote device to control a single sorbent recharger by a doctor or clinician.

FIG. 4 shows use of two different remote devices to control a single sorbent recharger by both a patient and a doctor or clinician.

FIG. 5 shows use of a single remote device to control multiple sorbent rechargers.

FIG. 6 shows use of a remote device to control multiple sorbent rechargers in a clinic setting.

FIG. 7 shows a non-limiting embodiment of components that can be included in a remote device.

FIG. 8 is a flow chart showing steps for establishing communication between a sorbent recharger and a remote device.

FIG. 9 is a flow chart showing steps for monitoring an ongoing recharging process.

FIG. 10 is a flow chart showing steps for controlling a recharging process with a remote device.

FIG. 11 is a flow chart showing the steps for viewing and monitoring configuration information of the sorbent recharger.

FIG. 12 is a flow chart showing steps for changing sorbent recharger configuration parameters.

FIG. 13 is a flow chart illustrating steps for remotely running diagnostic tests on the sorbent recharger using a remote device.

### DETAILED DESCRIPTION

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

An “action to initiate a diagnostic test” refers to an act that must be performed by a user to begin a diagnostic test on a component.

An “action to initiate a function” is an act that must be performed by a user to begin a process or to begin a step in a process.

An “action to power off” a component is an act that must be performed by a user to shut down and turn off a component.

An “action to recover an alert” is an act that must be performed by a user or system in response to an alert indicating an alert before a process can be taken.

An “action to recover an error” is an act that must be performed by a user or system in response to an alert indicating an error or problem before a process can be taken.

An “an action to set or change a configuration parameter” is an act that must be performed by a user to either set one or more parameters to be used in recharging a sorbent material or to adjust one or more parameters to be used in recharging a sorbent material.

An “action to stop” a process refers to an act that is undertaken by a user to end or pause a process.

An “alert” is any tactile, visual, or audio cue indicating the state of a system or component.

An “audio signal” is a signal to a user of a device that uses sound.

The terms “authenticating” or to “authenticate” refer to a process by which a user or device is determined to have access to data or functions of a device in order to grant the user the ability to obtain the data or perform the functions.

“Authentication information” refers to information that identifies a specific component or user.

“Bluetooth” is a short-range wireless communication using UHF radio waves.

The terms “communication,” “communicate,” “communicating,” and the like can refer to the ability to transmit electronic data, instructions, information wirelessly, via direct electrical connection, or any other electrical transmission means between one or more components.

The term “comprising” includes, but is not limited to, whatever follows the word “comprising.” Use of the term indicates the listed elements are required or mandatory but that other elements are optional and may be present.

A “configuration parameter,” as used herein, refers to any parameter of a recharging process. Configuration parameters can include, but are not limited to, concentrations, temperature, flow rates, or volumes of one or more solutions used in recharging a sorbent material.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts, or features that do not affect the basic operation of the apparatus, structure or method described.

To “control” a process refers to a component or system that causes other components or systems to perform some action.

The term “data” can refer to any quantity, text, character, or symbol containing or representing information of any type. In general, a computer can perform operations on the data, and the data recorded and stored on any one of magnetic, optical, electrical, memory, or mechanical recording media, and transmitted in the form of digital electrical signals.

A “display” is a component of an interface in which a device can provide visual data to a user.

To “display” refers to a process of providing visual information to a user.

To “establish” communication means to perform some actions that allow data to be sent electronically between two components.

A “function necessary” to carry out a process or method refers to any step in the process or method without which the process or method would not proceed as intended.

The term “haptic signal” refers to a signal to a user of a device that uses the sense of touch of the user.

The term “input” refers to a component on a device through which data or instructions can be provided to the device.

The term “processor” or “processing unit” as used is a broad term and is to be given an ordinary and customary meaning to a person of ordinary skill in the art. The term refers without limitation to a computer system, state machine, processor, or the like designed to perform arithmetic or logic operations using logic circuitry that responds to and processes the basic instructions that drive a computer. In any embodiment of the first, second, third, and fourth invention, the terms can include ROM (“read-only memory”) and/or RAM (“random-access memory”) associated therewith.

The term “programmed,” when referring to a processor, can mean a series of instructions that cause a processor to perform certain steps.

The term “receiving” or “to receive” refers to the process of obtaining electronic information by any means.

“Recharging” refers to treating a sorbent material to restore the functional capacity of the sorbent material to put the sorbent material back into a condition for reuse or use in a new dialysis session. In some instances, the total mass, weight and/or amount of “rechargeable” sorbent materials remain the same. In some instances, the total mass, weight and/or amount of “rechargeable” sorbent materials change. Without being limited to any one theory of invention, the recharging process may involve exchanging ions bound to the sorbent material with different ions, which in some instances may increase or decrease the total mass of the system. However, the total amount of the sorbent material will in some instances be unchanged by the recharging process. Upon a sorbent material undergoing “recharging,” the sorbent material can then be said to be “recharged.”

The term “recharge status” refers to the current process being performed by a sorbent recharger, and can include information such as the time left in the process, previous steps in the process, or subsequent steps in the process.

A “remote device” is any device separate from other components in a system with which the remote device interacts.

A “request for action” refers to data indicating to a user that some action must be taken for a process to continue.

The term “reusable” refers to a component that can be used more than once. In certain embodiments, the component can be treated to allow reuse in between uses.

“Sorbent materials” are materials capable of removing specific solutes from solution, such as cations or anions.

A “sorbent module” means a discreet component of a sorbent cartridge. Multiple sorbent cartridge modules can be fitted together to form a sorbent cartridge of two, three, or more sorbent cartridge modules. The “sorbent cartridge module” or “sorbent module” can contain any selected materials for use in sorbent dialysis and may or may not contain a “sorbent material” or adsorbent, but less than the full complement of sorbent materials needed. In other words, the “sorbent cartridge module” or “sorbent module” generally refers to the use of the “sorbent cartridge module” or “sorbent module” in sorbent-based dialysis, e.g., REDY (REcirculating DYalysis), and not that a “sorbent material” that is necessarily contained in the “sorbent cartridge module” or “sorbent module.”

A “sorbent recharger” or “recharger” is an apparatus designed to recharge at least one sorbent material.

The terms “transmission,” “to transmit,” “transmitting,” and the like can refer to the ability to send electronic data, instructions, information wirelessly, via direct electrical connection, or any other electrical communication means between one or more components.

A “user” is any person that interacts with a system or components of the system.

A “wearable device” is a device designed to be worn on the body of a user, such as a watch or glasses.

“Wi-Fi” is a wireless communication protocol using radio waves.

“Wi-Fi direct” is a wi-fi connection between two devices without an intermediary access point.

“Wireless communication” refers to the ability to send data between two or more components without a direct electrical connection between the components.

“Zigbee” is a wireless mesh network standard operating in the radio spectrum.

### Remote Monitoring of a Sorbent Recharger

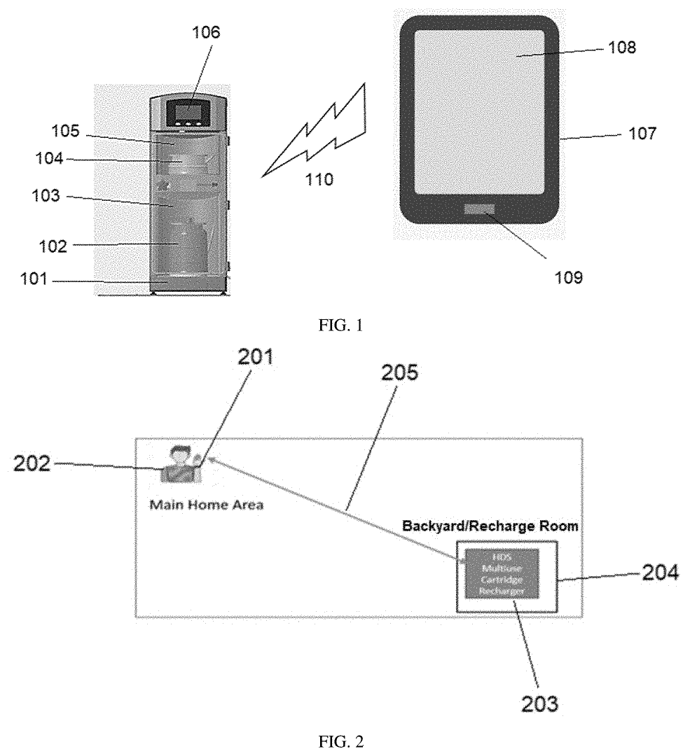

FIG. 1 illustrates the use of a remote device 107 for controlling at least one function of a sorbent recharger 101. The sorbent recharger 101 can be include a first receiving compartment 103 for receiving a reusable sorbent module 102 containing used sorbent material. In certain embodiments, the sorbent recharger 101 can include a second receiving compartment 105 for receiving a second reusable sorbent module 104 containing used sorbent material. The first sorbent module 102 and second sorbent module 104 can have the same or different sorbent materials. For example, both sorbent module 102 and sorbent module 104 can contain zirconium phosphate, both sorbent module 102 and sorbent module 104 can contain zirconium oxide, or one sorbent material can contain zirconium phosphate and the other can contain zirconium oxide.

To recharge the sorbent material in sorbent module 102 or sorbent module 104, recharge solutions must be passed through the sorbent modules. For example, zirconium phosphate can be recharged by passing solutions containing hydrogen and sodium ions through the material. The hydrogen and sodium ions in the recharge solution can exchange with potassium, calcium, magnesium, and ammoniums ions bound to the zirconium phosphate during use. The final ratio of sodium to hydrogen ions on the zirconium phosphate depends on the sodium concentration and pH of the recharge solution used, and can be controlled based on the needs of the dialysis system and user. Zirconium oxide can be recharged by passing a basic solution through the zirconium oxide, as well as a solution for disinfection, such as bleach.

To begin recharging sorbent materials in sorbent module 102 and/or sorbent module 104, a user must give a command to the sorbent recharger 101. Further, certain steps in the recharging process may require the user to confirm actions to be taken. Specific parameters used in recharging, such as the pH or sodium concentration of the recharge solution, flow rates, and temperature can be set by a user. In certain instances, error messages or alerts may need to be transmitted to a user and corrective action taken.

The sorbent recharger 101 can include a graphical user interface 106 to display messages to a user and receive input from the user. However, efficiency can be improved and the risk of cross-contamination when recharging sorbent modules used by multiple patients reduced if the direct contact between the users and the sorbent module 101 is reduced or eliminated.

A remote device 107 can be used to control the sorbent recharger 101. Although shown as a tablet type device, the remote device 107 can be any device that is remote or separate from the sorbent recharger 101. For example, the remote device 107 can be a wearable device, such as a watch or glasses, a phone, a tablet, a laptop, or a desktop computer. Any device capable of sending and receiving information to and from sorbent recharger 101 can be used.

The remote device 107 can include a processor (not shown), a display screen 108 and an input 109. In certain embodiments, the display screen 108 can be a touchscreen, making the input 109 optional. Additionally, the input 109 can be separated from the remote device. For example, a wearable device may have only a display screen or only an input, with a second remote device having the other.

The processor of the remote device 107 can be programmed to establish wireless communication with the sorbent recharger 101, as shown by signal 110. The mode of communication can be by any wireless means. For example, the system can use Bluetooth, Wi-Fi, Wi-Fi direct, Zigbee, or any other type of wireless communication technology. The remote device 107 can transmit device authentication information to the sorbent recharger 101 and receive recharger authentication information from the sorbent recharger 101. After authentication of the remote device 107 and sorbent recharger 101, the remote device 107 can control functions necessary for recharging the sorbent materials. For example, the remote device 107 can be used to provide an action to initiate a function in recharging the sorbent material, an action to recover an error on the sorbent recharger, an action to recover an alert from the sorbent recharger, an action to initiate recharging the sorbent material, an action to power off the sorbent recharger, an action to initiate one or more diagnostic tests, and/or an action to set or change one or more configuration parameters for the sorbent recharger. The remote device 107 can transmit the data to the sorbent recharger 101.

The remote device 107 can also receive data from the sorbent recharger 101. For example, the remote device 107 can receive one or more configuration parameters from the sorbent recharger 101, such as the pH, temperature, flow rate, volume, or solute concentration of a recharge solution. The remote device 107 can also receive a recharge status, an alert, and/or a request for action from the sorbent recharger 101. Data received from the sorbent recharger 101 can be displayed on the display screen 108 of the remote device 107. Alternatively, or additionally, the remote device 107 can be programmed to provide an audio and/or a haptic signal based on data received from the sorbent recharger 101.

FIG. 2 illustrates use of a single remote device 201 to control a single sorbent recharger 203 by a user 202 as may be done for home dialysis. The remote device 201 can establish communication with the sorbent recharger 203, as shown by line 205. The sorbent recharger 203 can be kept in a dedicated room 204 or outdoor structure separate from the main home area to conserve space in the home and eliminate odors from the recharger chemicals. The user 202 can use the remote device 201 to control at least one function necessary for recharging sorbent materials in reusable sorbent modules. The communication between the remote device 201 and sorbent recharger 203 can be by Wi-Fi, Wi-Fi direct, Bluetooth, or any other methods. Bluetooth is particularly suitable for a single remote device 201 to control a single sorbent recharger 203, so long as the sorbent recharger 203 is within Bluetooth range of the remote device 201. Similarly, Wi-Fi direct is suitable for a single remote device 201 to control a single sorbent recharger 203, so long as the sorbent recharger 203 is within range of the remote device 201. If the sorbent recharger 203 is further away from the remote device 201, Wi-Fi can be used, with the signal traveling through a router and the internet (not shown).

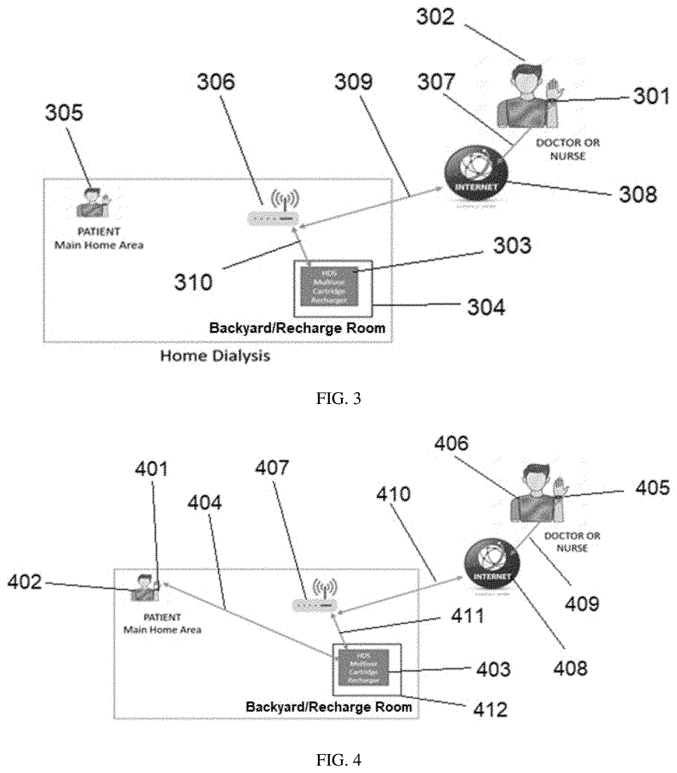

FIG. 3 illustrates use of a single remote device 301 to control a single sorbent recharger 303 by a doctor or health care professional 302 as may be done for home dialysis. The doctor or health care professional 302 can communicate with the sorbent recharger 303 using Wi-Fi technology. The remote device 301 can transmit and receive data through the internet 308 as shown by line 307. The sorbent recharger 303 can communicate through router 306 as shown by line 310. The router 306 can transmit and receive data from the internet 308 as shown by line 309. Using Wi-Fi, the doctor or health care professional 302 can control the sorbent recharger 303 through the internet 308. The patient 305 does not need to control any aspect of the recharging process other than to insert the sorbent modules into the sorbent recharger 303. As described, the sorbent recharger 303 can be kept in a dedicated room 304 or outdoor structure separate from the main home area to conserve space in the home and eliminate odors from the recharger chemicals.

FIG. 4 illustrates the use of multiple remote devices to control a single sorbent recharger 403 by both a user 402 and a doctor or health care professional 406. Similar to FIG. 2, the user 402 of FIG. 4 can control the sorbent recharger 403. The remote device 401 can establish communication with the sorbent recharger 403, as shown by line 404. The sorbent recharger 403 can be kept in a dedicated room 412 or outdoor structure separate from the main home area to conserve space in the home and eliminate odors from the recharger chemicals. The user 402 can use the remote device 401 to control at least one function necessary for recharging sorbent materials in reusable sorbent modules. The communication between the remote device 401 and sorbent recharger 403 can be by Wi-Fi, Wi-Fi direct, Bluetooth, or any other methods. Similarly, a doctor or health care professional 406 can use a second remote device 405 to control at least one function necessary for recharging sorbent materials. The doctor or health care professional 406 can communicate with the sorbent recharger 403 using Wi-Fi technology. The remote device 405 can transmit and receive data through the internet 408 as shown by line 409. The sorbent recharger 403 can communicate through router 407 as shown by line 411. The router 407 can transmit and receive data from the internet 408 as shown by line 410. Using Wi-Fi, the doctor or health care professional 406 can control the sorbent recharger 403 through the internet 408. In certain embodiments, the remote device 401 of the user 402 and the remote device 405 of the doctor or health care professional 406 can each perform the same functions. That is, both remote devices can control all aspects of recharging. Alternatively, the remote device 401 of the user 402 and the remote device 405 of the doctor or health care professional 406 can perform different functions. For example, the remote device 401 of the user 402 can control the starting of recharging, while remote device 405 of the doctor or health care professional 406 can set configuration parameters for the sorbent recharger 403. Any combination of functions can be allocated to each of the remote device 401 of the user 402 and the remote device 405 of the doctor or health care professional 406. Each remote device may be able to receive and view data from the sorbent recharger 403 and respond to any alerts or errors. In certain embodiments, the user 402 can generally control the recharging process, but the doctor or health care professional 406 can monitor the process to ensure proper recharging and receive any alerts or errors that may occur, allowing the doctor or health care professional 406 to intervene if necessary.

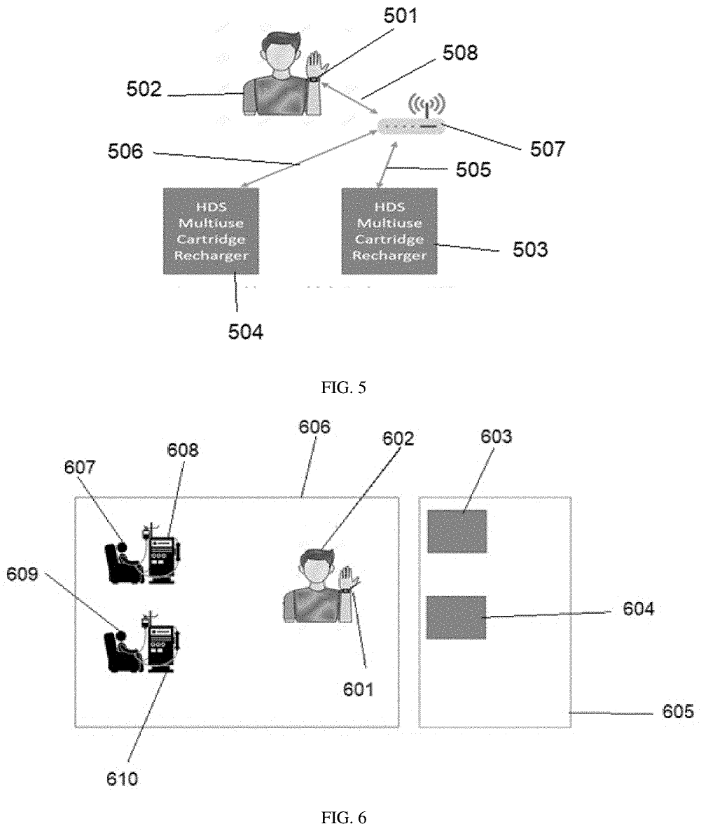

FIG. 5 illustrates the use of a remote device 501 to control multiple sorbent rechargers, as may be done by a doctor or health care professional 502 with multiple home dialysis patients. A remote device 501 can establish communication with a first sorbent recharger 503 and a second sorbent recharger 504. The remote device 501 can communicate through router 507 as shown by line 508. One of skill in the art will understand that the remote device 501 can communicate through the internet and multiple routers can be used. The router 507 can be in communication with the first sorbent recharger 503 as shown by line 505 and with the second sorbent recharger 504 as shown by line 506. The first sorbent recharger 503 and second sorbent recharger 504 can communicate with the remote device 501 through separate routers. The doctor or health care professional 502 can control each of the first sorbent recharger 503 and second sorbent recharger 504 to separately recharge different sorbent modules. Although shown as two sorbent rechargers 503 and 504, one of skill in the art will understand that any number of sorbent rechargers can communicate with a single or multiple remote devices. As described, in certain embodiments, the patient may also have a remote device (not shown) that can communicate with and optionally control the sorbent recharger specifically associated with that patient.

FIG. 6 shows use of a remote device 601 in a typical clinical setting. A doctor or clinician 602 may be responsible for multiple patients. For example, the doctor or clinician 602 can be simultaneously responsible for providing dialysis treatment to a first patient 607 using a first hemodialysis console 608 and a second patient 609 using a second hemodialysis console 610. At the same time, a sorbent module that was previously used with a third patient may be recharged using a first sorbent recharger 603 and a sorbent module previously used by a fourth patient may be recharged using a second sorbent recharger 604. As described, the first sorbent recharger 603 and second sorbent recharger 604 may be kept in a separate room 605 from the main dialysis clinic room 606. One of skill in the art will understand that any number of patients and rechargers can be included in the system under the control of doctor or clinician 602.

The infection status of the first patient 607, second patient 608, the third patient and the fourth patient may be different. Certain patients may be positive with Hepatitis A, Hepatitis B, Hepatitis C and other infections, with a single doctor or clinician 602 in charge of each patient. Every time that doctor or clinician 602 interacts with the first patient 607 or second patient 608 there is a chance of cross-contamination and spread of disease. The cross-contamination can include the sorbent modules that were previously used by the third and fourth patients. Each time the doctor or clinician 602 enters the sorbent recharger room 605 and performs a function with the first sorbent recharger 603 and second sorbent recharger 604 the risk of cross contamination increases, as the doctor or clinician 602 must touch the rechargers to perform functions or recover alerts and errors. The cross-contamination can include cross-contamination between patients when the doctor or clinician 602 touches sorbent modules used by one patient after interacting with a second patient, or vice versa. There is also a risk of chemical allergies due to the recharger chemicals in recharger room 605, as well as other contaminants such as bacteria. However, by using the remote device 601 to control the first sorbent recharger 603 and second sorbent recharger 604, the doctor or clinician 602 minimizes contact with the sorbent rechargers and used sorbent modules. The remote device 601 reduces the touch points necessary with each sorbent recharger. For example, the clinician can receive recharge status information remotely and control the sorbent rechargers remotely, reducing contact between various patients' sorbent modules, chemicals, and sorbent rechargers. Using a remote device 601 to control the sorbent rechargers also allows the functions to be performed more quickly, reducing the down time of equipment, as the monitoring and control functions can be done without the doctor or clinician 602 being physically present.

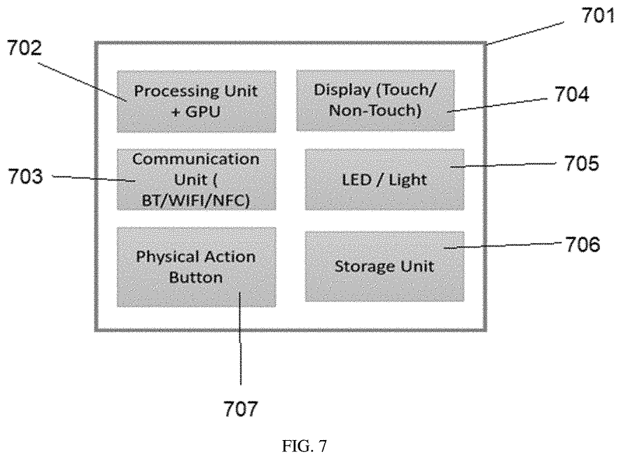

FIG. 7 is a non-limiting embodiment of components that can be used with in a remote device 701. The remote device 701 can include a processor 702 including a graphics processing unit. The processor 702 can be programmed to allow the remote device 701 to control and interact with one or more sorbent rechargers. The remote device 701 can also include a communication unit 703. The communication unit 703 establishes communication with a sorbent recharger and sends and receives data from the sorbent recharger. The communication unit can use any type of communication, including Bluetooth, Wi-Fi, or near field communication. In certain embodiments, the remote device 701 can include a physical action button 704. The physical action button 704 allows the user to cause the remote device 701 to take an action. The remote device 701 can also include a display 705. The display 705 provides information to the user. The display 705 can include lights or a screen. If a touch screen is used, in certain embodiments, the physical action button 704 is not necessary. In certain embodiments, the remote device 701 can include an LED or light 706. However, if the display 705 can provide all information necessary to the user, a separate LED or light 706 may not be necessary. The remote device 701 can also include a storage unit 707 for storing information. The storage unit 707 can store applications or instructions programmed to control the remote device 701. The storage unit 707 can also store data concerning the sorbent recharge and patient information. One of skill in the art will understand that additional components can be included in the remote device 701. For example, speakers can be included to provide an audio signal to the user. Components can also be included to provide haptic signals to the user.

### Use of a Remote Device

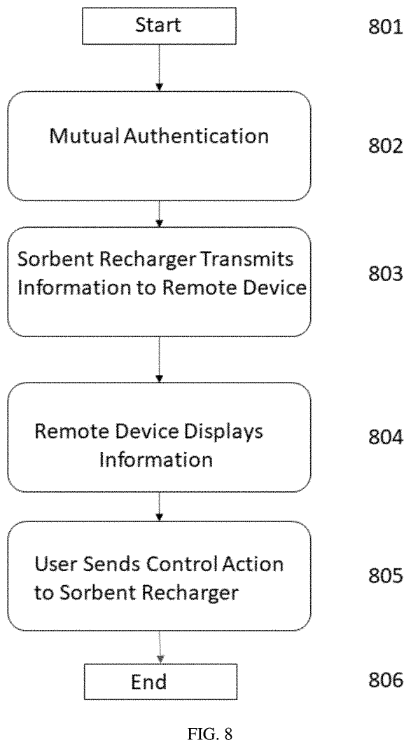

FIG. 8 is a flow chart showing steps that can be followed to establish communication between a sorbent recharger and a remote device, and to control the sorbent recharger with the remote device. The process can start in step 801. In step 802, the sorbent recharger and remote device can mutually authenticate each other. The remote device can transmit authentication information to the sorbent recharger and the sorbent recharger can transmit authentication information to the remote device. The remote device and sorbent recharger can check the received authentication information to ensure that the user is establishing communication with the correct sorbent recharger and that the user has access to the sorbent recharger.

In step 803, the sorbent recharger can transmit certain information to the remote device, which can display that information to the user in step 804. The information can include patient information, sorbent recharger information, recharger configuration parameters, or any other information. In step 805, the user can send a control action to the sorbent recharger using the remote device. For example, the user can start a recharging process, set one or more sorbent recharger configuration parameters, respond to an alert or error, turn off the sorbent recharger, or any other action. In step 806, the process can end.

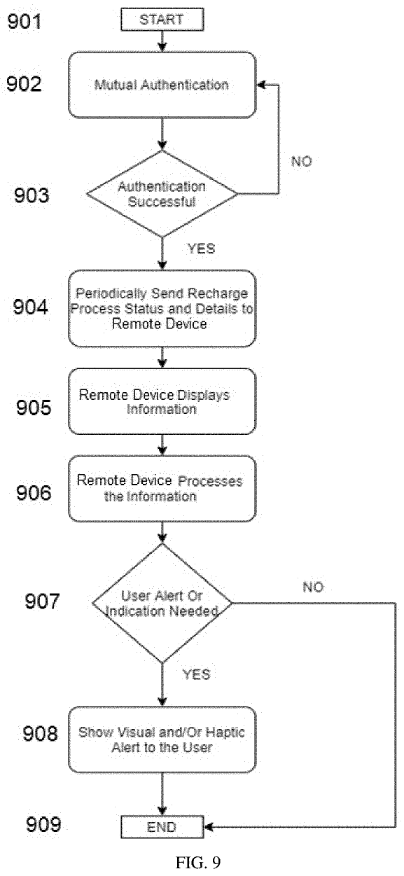

FIG. 9 is a flow chart showing the use of a remote device in monitoring an ongoing recharging process. The process can start in step 901. In step 902 the sorbent recharger and remote device can be mutually authenticated as described with respect to FIG. 8. In step 903 the sorbent recharger and remote device can independently determine whether the mutual authentication was successful. If not, the process can start over in step 901, and a message indicating the problem can be sent to the user.

If mutual authentication is successful, the sorbent recharger can begin periodically transmitting recharge status information to the remote device in step 904. The recharge status information can be transmitted at a fixed interval, such as three seconds, or whenever some action is needed. The remote device can display the information received from the sorbent recharger in step 905 and process the information in step 906. One of skill in the art will understand that steps 905 and 906 can be reversed. In step 907, the remote device determines whether a user alert or indication is needed. If so, the alert can be provided in step 908 either visually on the remote device, or by an audio or haptic alert. Examples of alerts can include an action required by the user, critical information for the user, an indication of an error or alarm on the sorbent recharger, or any other information. Errors and alerts can include recharge process errors, such as when a step in the recharging process does not proceed as expected. Other errors can include a door error if the doors are not fully closed or locked, a water input error if the water source is not properly connected, a water quality error if the source water is not clean enough to use in recharging, an infection status error if the system determines the patient may have an ongoing infection, connection errors if any tubes are not properly connected, chemical errors if any chemical source is incorrect or the wrong concentration, or any other errors. The process can end in step 909.

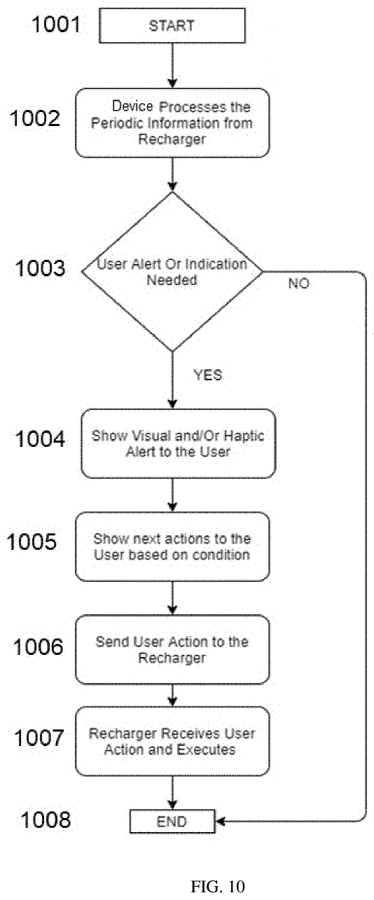

FIG. 10 is a flow chart showing steps for controlling a recharging process with a remote device. The process can start in step 1001 after mutual authentication of the remote device and sorbent recharger, and after the sorbent recharger has begun transmitting periodic recharge status information to the remote device. As described with respect to FIG. 9, the remote device can process the information received from the sorbent recharger in step 1002 and determine whether a user alert or indication is needed in step 1003. If so, the remote device can provide the alert or signal to the user in step 1004 with a request for action to be taken. The remote device can show the user the actions necessary to take based on the alert in step 1005. The actions necessary can include actions to recover an error on the sorbent recharger, actions to recover an alert on the sorbent recharger, actions to confirm a step and continue the recharging process, an action to stop the recharging process due to some error, and/or an action to power off the sorbent recharger as a safety action. The user can transmit instructions to the sorbent recharger using the remote device in step 1006. The sorbent recharger can receive the instructions in step 1007 and execute the actions provided in the instructions. The process can end in step 1008.

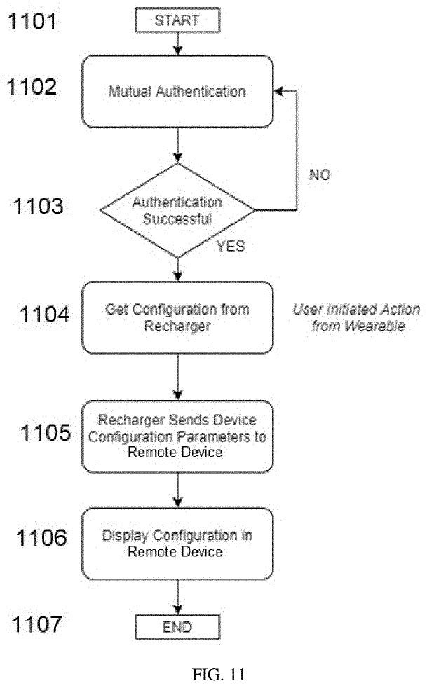

FIG. 11 is a flow chart showing the steps for viewing and monitoring configuration information of the sorbent recharger. The process can start in step 1101. In step 1102, the sorbent recharger and remote device can mutually authenticate each other. In step 1103 the sorbent recharger and remote device can independently determine whether the mutual authentication was successful. If not, the process can start over in step 1101, and a message indicating the problem can be sent to the user.

If mutual authentication is successful, the user can initiate an action to get the sorbent recharger configuration information in step 1104. The remote device can include an option for the user to request the configuration information. Alternatively, the sorbent recharger can automatically transmit the configuration information to the user without the need for a specific request. In step 1105, the sorbent recharger can send the device configuration information to the remote device. The remote device can display the configuration information to the user in step 1106. The process can end in step 1107.

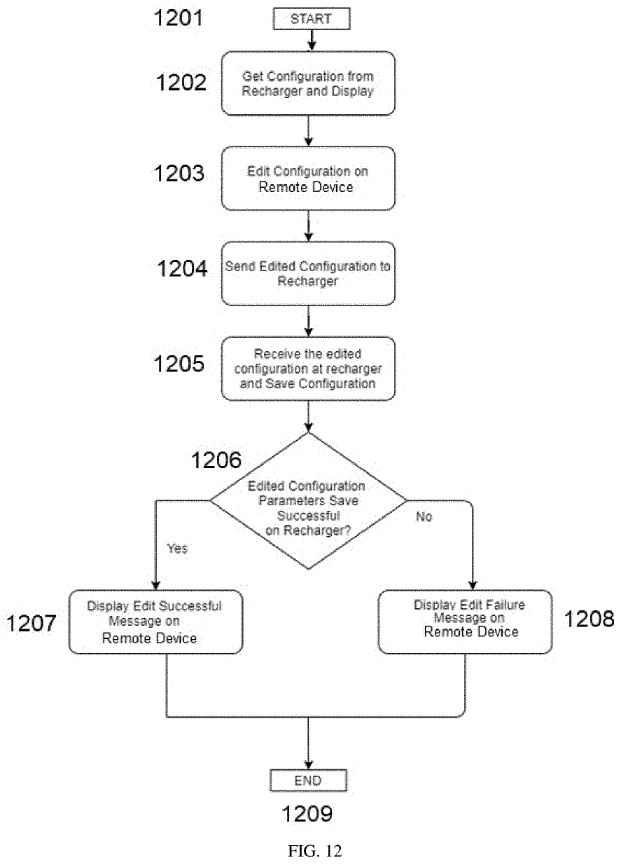

FIG. 12 is a flow chart showing steps for changing sorbent recharger configuration parameters. The process can start in step 1201 after mutual authentication of the remote device and sorbent recharger. In step 1202, the remote device can receive and display the sorbent recharger configuration information as described with respect to FIG. 11. In step 1203, the user can edit the configuration parameters on the remote device. The edited configuration parameters can be transmitted to the sorbent recharger in step 1204. The sorbent recharger can receive the edited configuration parameters and save the changes in step 1205. In step 1206, the remote device can determine whether the edited configuration parameters were successfully saved by the sorbent recharger. If the edited configuration parameters were successfully saved by the sorbent recharger, the remote device can display a success message in step 1207. If the edited configuration parameters were note successfully saved by the sorbent recharger, the remote device can display a failure message in step 1208. Edited configuration parameters may not be saved due to a communication interruption or because the edited configuration parameters are rejected. If the edited configuration parameters would result in an unsafe or ineffective recharging process, the sorbent recharger may fail to save the edited configuration parameters. The process can end in step 1209.

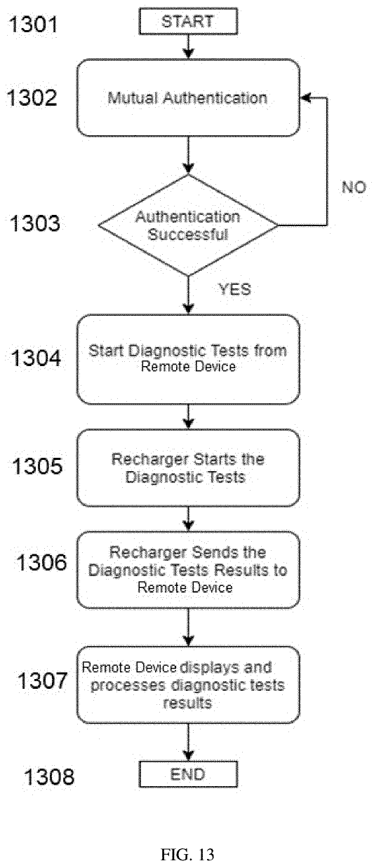

FIG. 13 is a flow chart illustrating steps for remotely running diagnostic tests on the sorbent recharger using a remote device. The process can start in step 1301. In step 1302 the sorbent recharger and remote device can be mutually authenticated. In step 1303 the sorbent recharger and remote device can independently determine whether the mutual authentication was successful. If not, the process can start over in step 1301, and a message indicating the problem can be sent to the user. In step 1304 the user can select an option on the remote device to start a diagnostic test. In step 1305, the sorbent recharger can receive instructions to start the diagnostic test from the remote device and begin the diagnostic test. In step 1306, the sorbent recharger can send the results of the diagnostic test to the remote device. The remote device can display the results to the user instep 1307. The process can end in step 1308.

One skilled in the art will understand that various combinations and/or modifications and variations can be made in the described systems and methods depending upon the specific needs for operation. Various aspects disclosed herein may be combined in different combinations than the combinations specifically presented in the description and accompanying drawings. Moreover, features illustrated or described as being part of an aspect of the disclosure may be used in the aspect of the disclosure, either alone or in combination, or follow a preferred arrangement of one or more of the described elements. Depending on the example, certain acts or events of any of the processes or methods described herein may be performed in a different sequence, may be added, merged, or left out altogether (e.g., certain described acts or events may not be necessary to carry out the techniques). In addition, while certain aspects of this disclosure are described as performed by a single module or unit for purposes of clarity, the techniques of this disclosure may be performed by a combination of units or modules associated with, for example, a medical device.

What is claimed is:

1. A system, comprising:
at least one sorbent recharger; and
at least one remote device in communication with the at least one sorbent recharger;
the at least one remote device programmed to control at least one function necessary for the sorbent recharger to recharge a sorbent material in a reusable sorbent module.

2. The system of claim 1, wherein the at least one remote device is a wearable device.

3. The system of claim 1, further comprising at least a second sorbent recharger in communication with the at least one remote device; the at least one remote device programmed to control at least one function necessary for the second sorbent recharger to recharge a sorbent material in a second reusable sorbent module.

4. The system of claim 1, further comprising at least a second remote device in communication with the at least one sorbent recharger; the second remote device programmed to control at least one function necessary for the at least one sorbent recharger to recharge the sorbent material.

5. The system of claim 1, further comprising at least a second remote device and a second sorbent recharger; the second the second remote device programmed to control at least one function necessary for the second sorbent recharger to recharge a sorbent material in a second reusable sorbent module.

6. The system of claim 5, wherein each remote device is in communication with each sorbent recharger; wherein each remote device is programmed to control at least one function necessary for each sorbent recharge to recharge the sorbent material in each reusable sorbent module.

7. A method performed by a remote device; the method comprising steps of:
establishing communication between the remote device and at least one sorbent recharger; and
controlling at least one function necessary for the at least one sorbent recharger to recharge a sorbent material in a reusable sorbent module.

8. The method of claim 7, further comprising the step of authenticating the sorbent recharger and transmitting remote device authentication information to the recharger.

9. The method of claim 7, further comprising the step of displaying data from the sorbent recharger on a display.

10. The method of claim 9, wherein the data received from the sorbent recharger comprises one or more of: a recharge status, an alert, a request for action, and/or recharger configuration information.

11. The method of claim 7, further comprising the step of providing an audio and/or a haptic signal based on data received from the sorbent recharger.

12. The method of claim 7, further comprising the step of receiving data from a user and transmitting the data to the sorbent recharger.

13. The method of claim 12, the data from the user comprises at least one of: an action to recover an error on the sorbent recharger, an action to recover an alert from the sorbent recharger, an action to initiate a function in recharging the sorbent material, an action to stop recharging the sorbent material, an action to power off the sorbent recharger, an action to initiate one or more diagnostic tests, and/or an action to set or change one or more configuration parameters for the recharger.

14. The method of claim 7, further comprising the steps of:
establishing communication between the remote device and at least a second sorbent recharger; and
controlling at least one function necessary for the second sorbent recharger to recharge a sorbent material in a reusable sorbent module.

15. A remote device; the remote device comprising a processor programmed to:
establish wireless communication with at least a first sorbent recharger; and
to control at least one function necessary for the sorbent recharger to recharge a sorbent material in a reusable sorbent module.

16. The remote device of claim 15, wherein the remote device is a wearable device.

17. The remote device of claim 15, the remote device programmed to communicate with the sorbent recharger via Bluetooth, Wi-Fi, Wi-Fi direct, or Zigbee.

18. The remote device of claim 15, further comprising a display, the remote device programmed to display data received from the sorbent recharger on the display.

19. The remote device of claim 18, wherein the remote device is programmed to receive one or more configuration parameters from the sorbent recharger and to display the one or more configuration parameters on the display.

20. The remote device of claim 18, wherein the data received from the sorbent recharger comprises recharge status, an alert, and/or a request for action.

21. The remote device of claim 15, further comprising an input, the remote device programmed to transmit data from the input to the sorbent recharger.

22. The remote device of claim 21, wherein the data from the input comprises at least one of: an action to recover an error on the sorbent recharger, an action to recover an alert from the sorbent recharger, an action to initiate a function in recharging the sorbent material, an action to stop recharging the sorbent material, an action to power off the sorbent recharger, an action to initiate one or more diagnostic tests, and/or an action to set or change one or more configuration parameters for the sorbent recharger.

23. The remote device of claim 15, the remote device programmed to establish wireless communication and control at least one function of at least a second sorbent recharger.

24. The remote device of claim 15, the remote device programmed to receive sorbent recharger authentication information from the sorbent recharger and to transmit remote device authentication information to the recharger.

25. The remote device of claim 15, the remote device programmed to provide an audio and/or a haptic signal based on data received from the sorbent recharger.

26. The remote device of claim 25, wherein the data received from the recharger comprises one or more alerts.
